(19) United States
(12) Patent Application Publication
(10) Pub. No.: US 2014/0350657 A1
(43) Pub. Date: Nov. 27, 2014

(54) EXPANDABLE MEDICAL DEVICE WITH AN END STRUCTURE HAVING A TRANSITIONAL SURFACE

(71) Applicant: Cook Medical Technologies LLC, Bloomington, IN (US)

(72) Inventor: Joshua P. Headley, Ellettsville, IN (US)

(73) Assignee: Cook Medical Technologies LLC, Bloomington, IN (US)

(21) Appl. No.: 14/281,071

(22) Filed: May 19, 2014

Related U.S. Application Data
(60) Provisional application No. 61/825,697, filed on May 21, 2013.

(51) Int. Cl. A61F 2/82

(52) U.S. Cl. CPC A61F 2/82
USPC 623/1.15

(57) ABSTRACT

An expandable intraluminal medical device is provided. The medical device is provided with end structures that have transitional surfaces defined by two angled planes. The transitional surfaces may be useful in reducing scraping between the expandable device and a restraining sheath used for delivering the medical device.

This application claims priority to U.S. Provisional Application No. 61/825,697, filed May 21, 2013, which is hereby incorporated by reference herein.

### BACKGROUND

The present invention relates generally to medical devices and more particularly to an intraluminal medical device.

Intraluminal medical devices are used by physicians to treat numerous conditions using minimally invasive procedures. Examples of intraluminal medical devices include stents, stent-grafts, filters, valves, etc. Stents are one type of intraluminal medical device that have become especially common. Stents are used to treat various organs, including the vascular system, colon, biliary tract, urinary tract, esophagus, trachea and the like. For example, stents are most commonly used to treat blockages, occlusions, narrowing ailments and other similar problems that restrict flow through a passageway. However, stents are also used in other treatments as well, such as the treatment of aneurysms. Stents have been shown to be useful in treating various vessels throughout the vascular system, including both coronary vessels and peripheral vessels (e.g., carotid, brachial, renal, iliac and femoral). In addition, stents have been used in other body vessels as well, such as the digestive tract.

Stents have become a popular alternative for treating a variety of medical conditions because stenting procedures are considerably less invasive than conventional procedures. As an example, stenosis of the coronary arteries was traditionally treated with bypass surgery. In general, bypass surgery involves splitting the chest bone to open the chest cavity and grafting a replacement vessel onto the heart to bypass a blocked, or stenosed, artery. However, coronary bypass surgery is a very invasive procedure that is risky and requires a long recovery time for the patient. By contrast, stenting procedures typically involve inserting a stent delivery system into a patient through a small access site and threading the delivery system through passageways in the patient's body to the desired treatment site. For example, stent delivery systems are commonly threaded through arteries in a patient's body in order to treat an interior region of the artery without needing to perform open surgery on the patient.

Many different types of stents and stenting procedures are possible. In general, however, stents are typically designed as tubular support structures that may be inserted percutaneously and transluminally through a body passageway. Traditionally, stents have been made from a metal or other synthetic material with a series of radial openings extending through the support structure of the stent to facilitate compression and expansion of the stent. Although stents may be made from many types of materials, including non-metallic materials, common examples of metallic materials that may be used to make stents include stainless steel, nitinol, cobalt-chrome alloys, amorphous metals, tantalum, platinum, gold and titanium. Typically, stents are implanted within a passageway by positioning the stent within the area to be treated and then expanding the stent from a compressed diameter to an expanded diameter. The ability of a stent to expand from a compressed diameter makes it possible to thread the stent to the area to be treated through various narrow body passageways while the stent is in the compressed diameter. Once the stent has been positioned and expanded at the area to be treated, the tubular support structure of the stent contacts and radially supports the inner wall of the passageway. As a result, the implanted stent mechanically prevents the passageway from narrowing and keeps the passageway open to facilitate fluid flow through the passageway. Where a graft is attached to the wall of a stent, the graft may serve to seal the exterior of the stent-graft from the inner lumen of the stent. For example, this may be useful in treating various types of aneurysms. A graft layer may also be useful in promoting tissue ingrowth and for drug elution.

Stents can generally be characterized as either balloon-expandable or self-expanding. However, stent designs and implantation procedures vary widely. For example, although physicians often prefer particular types of stents for certain types of procedures, the uses for balloon-expandable and self-expanding stents sometimes overlap and procedures related to one type of stent may be adapted to other types of stents in certain situations.

Balloon-expandable stents are commonly used to treat stenosis of the coronary arteries. Usually, balloon-expandable stents are made from ductile materials that plastically deform relatively easily. In the case of stents made from metal, 316L stainless steel that has been annealed is a common choice for this type of stent. One procedure for implanting balloon-expandable stents involves mounting the stent circumferentially on the balloon of a balloon-tipped catheter and threading the catheter through a vessel passageway to the area to be treated. Once the balloon is positioned at the narrowed portion of the vessel to be treated, the balloon is expanded by pumping saline through the catheter to the balloon. As a result, the balloon simultaneously dilates the vessel and radially expands the stent within the dilated portion. The balloon is then deflated and the balloon-tipped catheter is retracted from the passageway. This leaves the expanded stent permanently implanted at the desired location. Ductile metal lends itself to this type of stent since the stent may be compressed by plastic deformation to a small diameter when mounted onto the balloon. When the balloon is later expanded in the vessel, the stent is once again plastically deformed to a larger diameter to provide the desired radial support structure. Traditionally, balloon-expandable stents have been more commonly used in coronary vessels than in peripheral vessels because of the deformable nature of these stents. One reason for this is that peripheral vessels tend to experience frequent traumas from external sources (e.g., impacts to a person's arms, legs, etc.) which are transmitted through the body's tissues to the vessel. In the case of peripheral vessels, there is an increased risk that an external trauma could cause a balloon-expandable stent to once again plastically deform in unexpected ways with potentially severe and/or catastrophic results. In the case of coronary vessels, however, this risk is minimal since coronary vessels rarely experience traumas transmitted from external sources.

Self-expanding stents are increasingly used and accepted by physicians for treating a variety of ailments. Self-expanding stents are usually made of shape memory materials or other elastic materials that act like a spring. Typical metals used in this type of stent include nitinol and 304 stainless steel. A common procedure for implanting a self-expanding stent involves a two-step process. First, the narrowed vessel portion to be treated is dilated with a balloon but without a stent mounted on the balloon. Second, a stent is implanted into the dilated vessel portion. To facilitate stent implantation, the stent is installed on the end of an inner catheter in a compressed, small diameter state and is usually retained in the small diameter by inserting the stent into a restraining sheath at the end of the catheter. The stent is then guided to the balloon-dilated portion and is released from the inner catheter by pulling the restraining sheath away from the stent. Once released from the restraining sheath, the stent radially springs outward to an expanded diameter until the stent contacts and presses against the vessel wall. Traditionally, self-expanding stents have been more commonly used in peripheral vessels than in coronary vessels due to the shape memory characteristic of the metals that are used in these stents. One advantage of self-expanding stents for peripheral vessels is that traumas from external sources do not permanently deform the stent. Instead, the stent may temporarily deform during an unusually harsh trauma but will spring back to its expanded state once the trauma is relieved. Self-expanding stents, however, are often considered to be less preferred for coronary vessels as compared to balloon-expandable stents. One reason for this is that balloon-expandable stents can be precisely sized to a particular vessel diameter and shape since the ductile metal that is used can be plastically deformed to a desired size and shape. In contrast, self-expanding stents are designed with a particular expansible range. Thus, after being implanted, self-expanding stents continue to exert pressure against the vessel wall.

Stents and other intraluminal medical devices may have various types of structures that allow the stent to be compressed for delivery into a patient's body and expand at the desired treatment site. The structure that allows the stent to compress and expand may also be formed in a variety of ways. For example, a stent structure may be made by bending a wire in a series of bends and straight sections around a cylindrical mandrel. Another common method for making a stent structure is to cut through the wall of a metal cannula with a laser to form an integral hollow cylinder with a series of bends and straight sections. Those of ordinary skill in the art readily recognize that these are only a few examples of the types and methods of making stents that are possible.

Intraluminal medical devices often have radiopaque markers or other enlarged structures at one or both ends of the device. In the case of a radiopaque marker, a material that blocks electromagnetic radiation, such as X-ray, is bonded to the marker. This is useful on intraluminal medical devices because as explained above the device is typically delivered through internal passages of a patient where the physician cannot directly see the device or the treatment site. However, radiopaque markers allow the physician to see the location of the device using an X-ray machine. Conventional radiopaque markers are also often used as an end surface to push on the device while loading the device into a delivery system or while releasing the device from the delivery system at the treatment site. Thus, radiopaque markers are often used for multiple purposes. However, where a particular intraluminal medical device does not require radiopaque markers at the ends of the device, other structures that are wider along the circumference of the device than the width of the members that make up the expandable structure may be desirable to provide a pushing surface or for other desired reasons.

### SUMMARY

An intraluminal medical device is described with end structures. The end structures have an end surface that faces away from an expandable structure of the device. A transitional surface is provided between the end surface and the outer surface which is disposed between two angled planes. The inventions herein may also include any other aspect described below in the written description, the claims, or in the attached drawings and any combination thereof.

### BRIEF DESCRIPTION OF SEVERAL VIEWS OF THE DRAWINGS

The invention may be more fully understood by reading the following description in conjunction with the drawings, in which:

FIG. 1 is a perspective view of a conventional self-expanding stent;

FIG. 2 is a perspective view of the distal end of the stent of FIG. 1;

FIG. 3 is a side view of a self-expanding stent with transitional surfaces on the end structures;

FIG. 4 is a perspective view of the distal end of the stent of FIG. 3;

FIG. 5 is a perspective view of the distal end of another stent with transitional surfaces on the end structures;

FIG. 6 is a perspective view of the distal end of another stent with transitional surfaces on the end structures; and

FIG. 7 is a cross-sectional view of one of the end structures.

### DETAILED DESCRIPTION

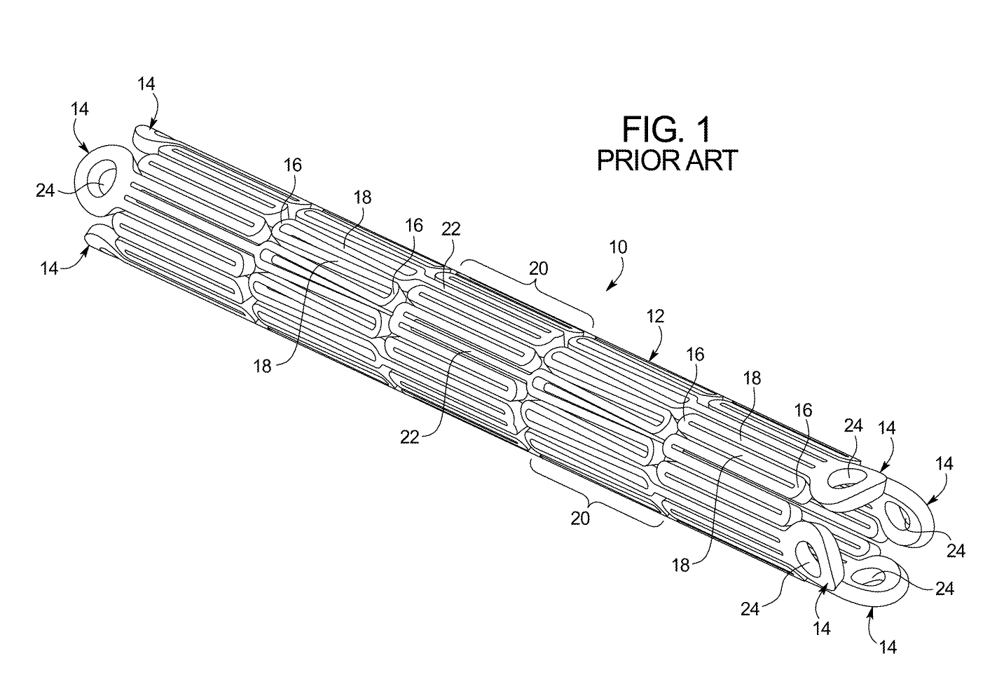

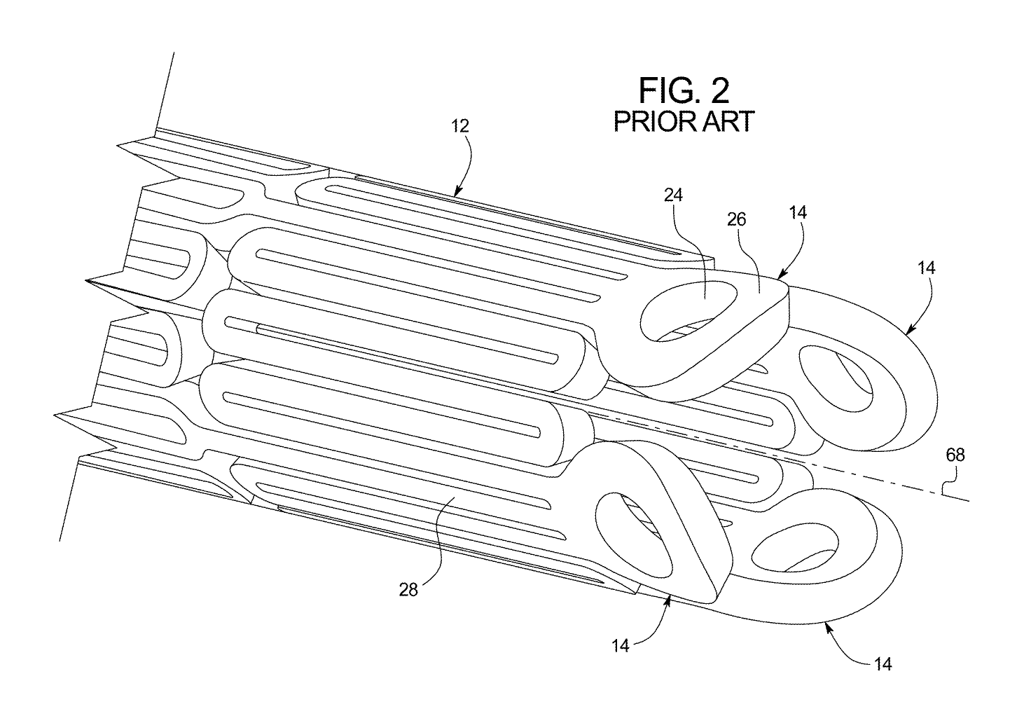

Referring now to the figures, and particularly to FIGS. 1-2, a conventional self-expanding stent 10 is shown. As shown, the stent 10 has an expandable structure 12 and end structures 14 attached to both ends of the expandable structure 12. The expandable structure 12 is shown in FIGS. 1 and 2 in the delivery configuration in which the stent 10 is compressed into a small diameter state suitable for being loaded into a delivery system and being threaded intraluminally through a patient's body. As shown, the expandable structure 12 may have a series of bends 16 that interconnect a series of straight sections 18, or struts 18. The bends 16 and struts 18 may be arranged in a series of rings 20 that are interconnected with longitudinal connectors 22. In the delivery configuration, as shown, the struts 18 may be positioned generally parallel and close to each other. In contrast, in the expanded configuration, the bends 16 allow the struts 18 to expand away from each other at an angle from each bend 16 so that the rings 20 expand diametrically.

Although the expandable structure 12 may have a variety of structures that are compressible and expandable and may be made in a number of ways, the expandable structure 12 is preferably cut with a laser from a metal cannula. Thus, the expandable structure 12 may be one integral structure. As illustrated in more detail in FIG. 2, the cross-section of each of the members of the expandable structure 12 is generally rectangular since the structure in this embodiment has been cut from a tube with a laser directed perpendicularly toward the axis of the stent 10. Because of the orientation of the laser and the rotation of the cannula during cutting, the lateral side surfaces of the struts 18 and the end structures 14 may have a slight inward taper toward the axis of the stent 10.

The end structures 14 are preferably evenly spaced around the circumference of the expandable structure 12. For example, as shown, the stent 10 may have four end structures 14 at each end of the expandable structure 12. If the end structures 14 are only desired on the distal end (i.e., the end that is released first from the delivery system), the end structures 14 could be provided only on the distal end. Alternatively, the end structures 14 could be provided only on the proximal end if desired. Although the end structures 14 may have any shape that is desirable, preferably the end structures 14 have an elliptical circumference, and more preferably, have a round circumference. In the preferred embodiment, the end structures 14 are radiopaque markers 14. Thus, the end structures 14 are provided with radial holes 24 through each end structure 14. A radiopaque material like gold, tungsten or platinum may be bonded in the hole 24 to make the marker 14 radiopaque. Where the expandable structure 12 and the end structures 14 are cut with a laser from a cannula, the end structures 14 may be integral with the expandable structure 12. The outer surface 26 of each end structure 14 is preferably contiguous with the outer surface 28 of the expandable structure 12.

One problem with conventional self-expanding stents 10 like in FIGS. 1-2 is that the transitional surface 30 between the end surface 32 and outer surface 26 of each end structure 14 can scrape against the restraining sheath of the delivery system, both when loading the stent 10 into the restraining sheath and when releasing the stent 10 from the delivery system at the treatment site. As illustrated in FIGS. 1-2, the transitional surfaces 30 in a conventional stent 10 are generally sharp, and can even taper inward slightly around the sides of the end structures 14. Because the stent 10 is self-expanding, the expandable structure 12 exerts significant outward pressure against the restraining sheath during loading as the stent 10 is pushed into the restraining sheath in its compressed state. As a result, the sharp transitional surfaces 30 of the end structures 14 can dig into and scrape against the inner surface of the restraining sheath. This may damage the inner surface of the restraining sheath and may shear loose pieces from the restraining sheath. Similarly, when the stent 10 is released from the delivery system at a treatment site, the sharp transitional surfaces 30 of the end structures 14 at the distal end can scrape against the inner surface of the restraining sheath. While this may damage the restraining sheath and shear loose pieces of the restraining sheath as noted, scraping of the transitional surfaces 30 may also increase the deployment force needed to release the stent 10 from the deployment system, which can make it more difficult to accurately deploy the stent 10 at the desired treatment site. In addition, the sharp transitional surfaces 30 may also irritate the vessel wall after or during deployment. In conventional stents 10, these problems are typically addressed by electropolishing the stent 10 after it has been laser cut from a metal cannula. While electropolishing smooths out roughness and generally rounds sharp corners 30 like those depicted in FIGS. 1-2, electropolishing generally retains the original structure of the stent 10 and is limited to smoothing the basic shape of the stent 10. However, the benefits of electropolishing may not be sufficient for some stents 10 and delivery systems.

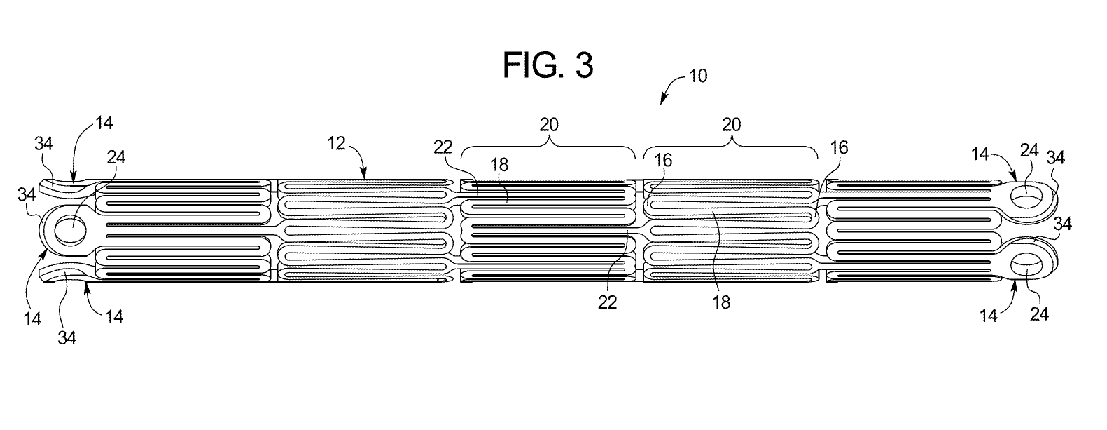

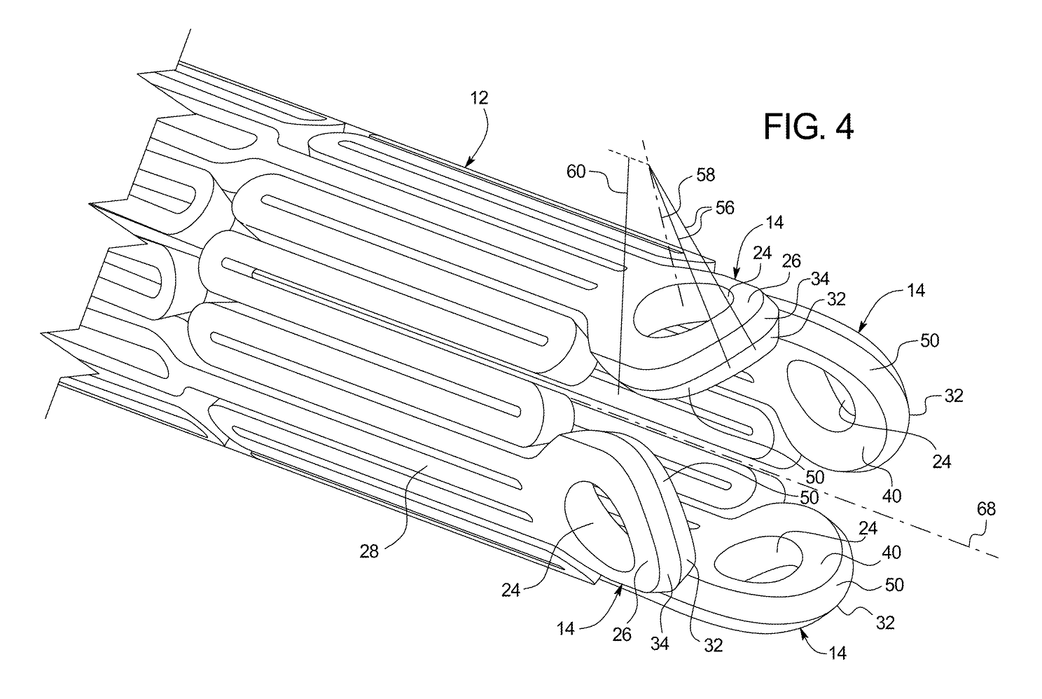

As shown in FIGS. 3-4, a self-expanding stent 10 may be provided with a transitional surface 34 between the end surface 32 of each end structure 14 and the outer surfaces 26 of the end structures 14 that is angled from the end surface 32 to the outer surface 26. The end surface 32 of each end structure 14 is generally considered to be the most distal or most proximal surface of the end structure 14 that faces away from the expandable structure 12. Thus, where the end structure 14 has a round circumference like in FIG. 4, the end surface 32 may be considered to be the apex of the round circumference which is intersected by a plane extending through the axis 68 of the stent 10 and bisecting the end structure 14. The end surface 32 is typically generally perpendicular to the outer surface 26 of the end structure 14.

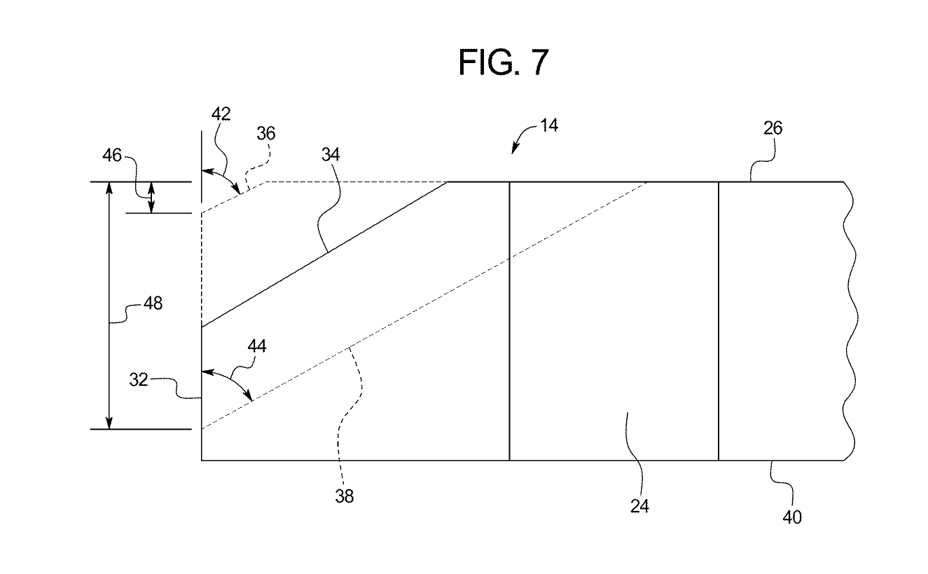

The transitional surface 34 may be defined by first and second planes 36, 38 as illustrated in FIG. 7. The first and second planes 36, 38 may be defined with respect to the thickness of the end structure 14 between the outer surface 26 and the inner surface 40 and by the angle of each plane 42, 44 with respect to a plane perpendicular to the axis of the stent 10. For example, the first plane 36 may be defined by a depth 46 at the end surface 32 that is 10% of the thickness from the outer surface 26, with the first plane 36 extending along a 10° angle 42 therefrom to the outer surface 26. The second plane 38 may be defined by a depth 48 at the end surface 32 that is 90% of the thickness from the outer surface 26, with the second plane 38 extending along a 60° angle 44 therefrom to the outer surface 26. (As shown in FIG. 7, the angles 42, 44 are measured between the planes 36, 38 and an extension line above the end surface 32.) The first and second planes 36, 38 define the transitional surface 34 by defining the boundaries of the transitional surface 34. Thus, the transitional surface 34 is disposed between the first and second planes 36, 38. As shown in FIG. 7, it is possible for the transitional surface 34 to intersect the hole 24 of the end structure 14 if desired. The transitional surface 34 may have any inclined shape within the first and second planes 36, 38 that is desirable; however it is preferred for the transitional surface 34 to be flat along an inclined angle. It is possible that the transitions to the transitional surface 34 may be rounded due to electropolishing or other finishing processes and the rounded transitions may fall outside the first and second planes 36, 38. However, such transitions are not considered to be part of the transitional surface 34 itself.

Preferably, the first and second planes 36, 38 may define a relatively narrow boundary for the transitional surface 34. For example, the first plane 36 may be defined by a depth 46 of 10% of the thickness and a 45° or 60° angle 42 therefrom. The first plane 36 may also be defined by a depth 46 of 30% of the thickness and a 10°, 45° or 60° angle 42 therefrom. The second plane 38 may be defined by a depth 48 of 90% of the thickness and a 45° or 30° angle 44 therefrom. The second plane 38 may also be defined by a depth 48 of 70% of the thickness and a 60°, 45° or 30° angle 44 therefrom.

While the transitional surface 34 as described above has been defined with respect to the most distal and/or proximal facing surface 32 of the end structures 14, the transitional surface 34 preferably extends at least partially around the sides 50 of the end structure 14. For example, the transitional surface 34 preferably extends at least 60° around the end structure 14 with the 60° range being centered around the end surface 32. However, as the transitional surface 34 extends further around the sides 50, or even rear facing surfaces (i.e., more than 180° centered around the end surface 32), there is less benefit to the transitional surface 34, at least in reducing scraping against the restraining sheath during loading and unloading. Therefore, the transitional surface 34 preferably does not extend around the entire end structure 14. Preferably, the transitional surface 34 extends at least 120° and not more than 240° around the end structure 14 with the 120°-240° range being centered around the end surface 32. Although the transitional surface 34 may also be provided on the struts 18 of the expandable structure 12, this is less desirable since the struts 18 typically have a relatively small width. Thus, preferably, the transitional surface 34 is only provided on the end structures 14. While the transitional surface 34 preferably remains within the boundaries defined above as the transitional surface 34 extends around the end structure 14, the slope of the transitional surface 34 may change as it extends around the end structure 14, and thus, may fall outside the boundaries defined above as long as the transitional surface 34 is within the boundaries at the end surface 32. For example, it may be desirable for the transitional surface 34 to become more vertical (i.e., be defined by a smaller angle) as the transitional surface 34 extends around the sides 50 of the end structure 14. This may allow the transitional surface 34 to gradually transition to the generally vertical side surfaces 50 of the end structure 14 at a location that is about 180° centered from the end surface 32 or at a location that is rearward facing (i.e., more than 180° centered from the end surface 34). However, the transitional surface 34 may provide a sufficient transition to the generally vertical side surfaces 50 while maintaining a constant slope around the end structure 14 by feathering out along a variety of different paths around the end structure 14.

As shown in FIGS. 4-6, the transitional surface 34, 52, 54 may extend around the end structure 14 in a number of different ways. In FIG. 4, the transitional surface 34 extends around about the front half of the end structure along a circular path that generally matches the circular diameter of the end structure 14. At about 180° centered about the end surface 32, the transitional surface 34 extends generally straight back towards the expandable structure 12 until the transitional surface 34 feathers out. As shown, the transitional surface 34 may have a generally constant angle as it extends around the end structure 14. Although the transitional surface 34 may be formed in a variety of ways, it may be particularly useful to cut the transitional surface 34 with a laser while the end structure 14 and expandable structure 12 are cut or in a subsequent cutting step with a laser. As shown FIG. 4, the path of the laser 56 may be centered on the axis 58 of the hole 24 extending through the end structure 14 along the front half of the end structure 14 and then may follow a path straight 60 back along the side 50 of the end structure 14. Preferably, a laser with an articulating head is used to achieve the angles and path that is desired for the transitional surface 34. In addition, the stent may be rotated as the transitional surface 34 is cut. Depending on the proximity of adjacent end structures 14, it is possible that the laser could contact and cut through portions of the adjacent end structures 14 when the expandable structure 12 is in the compressed configuration. If this would occur with a desired cutting path, the expandable structure 12 may be expanded at least partially when the transitional surface 34 is cut and then the stent 10 could be recompressed into the delivery configuration.

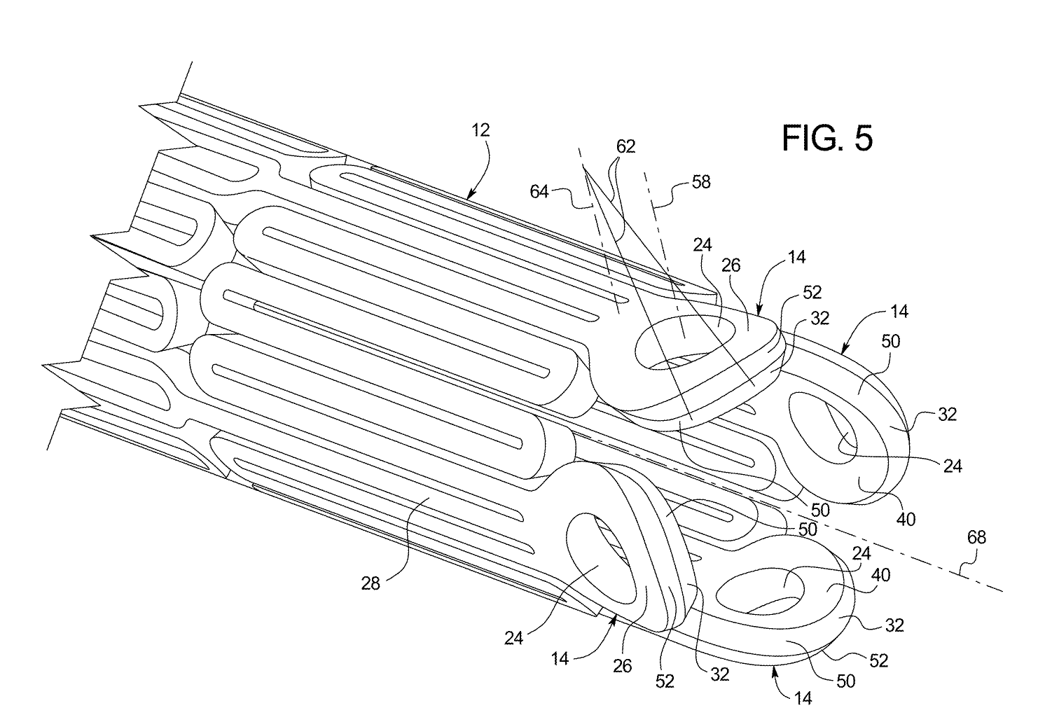

In FIG. 5, the transitional surface 52 may extend around the end structure 14 along a circular path that has a larger radius than the outer diameter of the end structure 14. Thus, the laser 62 may rotate around an axis 64 behind the axis 58 of the hole 24 through the end structure 14. As a result, the transitional surface 52 will feather out around the side 50 of the end structure 14 as the radius of the laser 62 passes away from the circumference of the end structure 14. As shown, the transitional surface 52 may have a generally constant angle as it extends around the end structure 14. In both FIGS. 4 and 5, the end structure 14 may have a generally elliptical circumference and the transitional surface 34, 52 may have a generally elliptical path around at least a portion of the end structure 14.

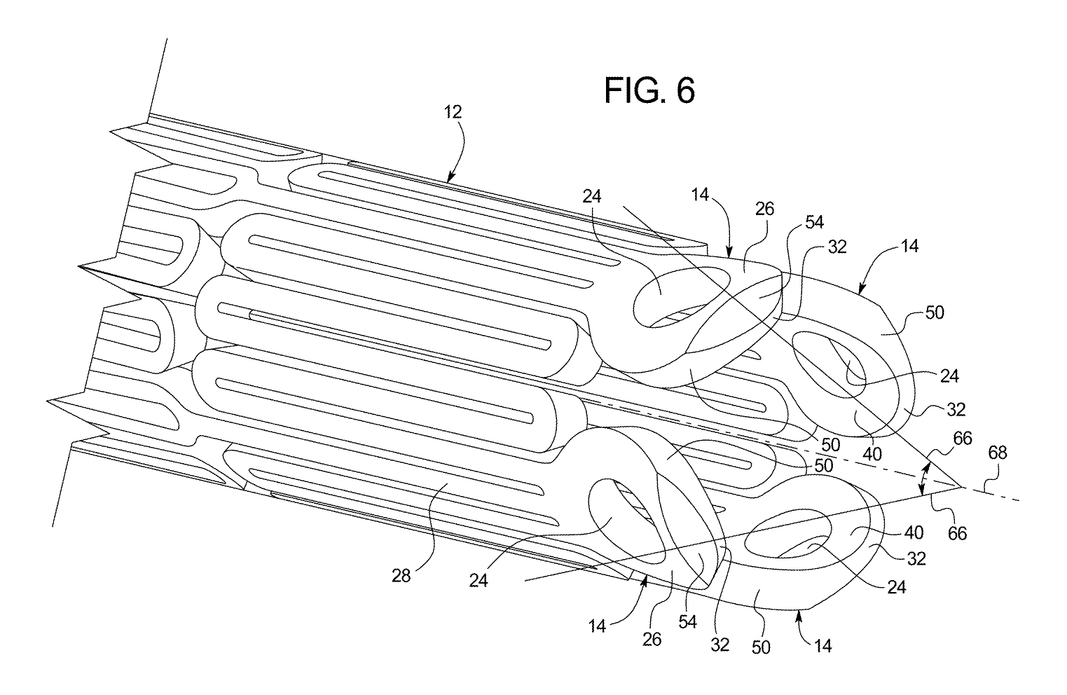

In FIG. 6, the transitional surface 54 may be formed by rotating the laser 66 around a generally constant angle with respect to the axis 68 of the expandable structure 12. For example, the laser 66 could be oriented at an angle to the stent 10, and the stent 10 could be rotated around its axis 68 while the laser 66 remains in place. It is possible that this method of cutting the transitional surface 54 may be easier since it could be done without an articulating head for the laser.

As is well-understood now, the transitional surfaces 34, 52, 54 on the end structures 14 may be advantageous in reducing scraping forces between the stent 10 and the restraining sheath during loading and unloading of the stent 10. It is also possible that the transitional surfaces 34, 52, 54 may provide a less traumatic transition between the stent 10 and the vessel wall during deployment of the stent 10 and while the stent 10 is implanted within the vessel.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

I claim:

1. An intraluminal expandable medical device, comprising:
an expandable structure comprising a delivery configuration and an expanded configuration; and
an end structure attached to an end of said expandable structure comprising an outer surface generally contiguous with an outer surface of said expandable structure, a thickness extending between said outer surface of said end structure and an inner surface of said end structure, and an end surface facing away from said expandable structure, said end surface being generally perpendicular to said outer surface of said end structure;
wherein said end structure comprises a transitional surface extending between said end surface and said outer surface of said end structure, said transitional surface being disposed between a first plane defined by 10% of said thickness from said outer surface of said end structure and a 10° angle extending therefrom to said outer surface of said end structure and a second plane defined by 90% of said thickness from said outer surface of said end structure and a 60° angle extending therefrom to said outer surface of said end structure.

2. The intraluminal expandable medical device according to claim 1, wherein said end structure is a radiopaque marker.

3. The intraluminal expandable medical device according to claim 1, wherein said intraluminal expandable medical device is a stent.

4. The intraluminal expandable medical device according to claim 3, wherein said stent is self-expanding.

5. The intraluminal expandable medical device according to claim 1, wherein said transitional surface extends at least 60° centered around said end surface around a circumference of said end structure.

6. The intraluminal expandable medical device according to claim 5, wherein said transitional surface extends at least 120° and not more than 240° centered around said end surface around a circumference of said end structure.

7. The intraluminal expandable medical device according to claim 1, wherein said end structure has a generally elliptical circumference and said transitional surface extends generally elliptically around at least a portion of said circumference of said end structure.

8. The intraluminal expandable medical device according to claim 7, wherein said transitional surface is defined by a generally constant angle as said transitional surface extends around said circumference of said end structure.

9. The intraluminal expandable medical device according to claim 7, wherein said end structure has a generally circular circumference and said transitional surface extends generally circularly around at least a portion of said circumference along a radius larger than a radius of said circumference.

10. The intraluminal expandable medical device according to claim 1, wherein said transitional surface is defined by a generally constant angle with respect to an axis of said expandable structure.

11. The intraluminal expandable medical device according to claim 1, wherein said first plane is defined by 30% of said thickness from said outer surface of said end structure.

12. The intraluminal expandable medical device according to claim 1, wherein said second plane is defined by 70% of said thickness from said outer surface of said end structure.

13. The intraluminal expandable medical device according to claim 1, wherein said first plane is defined by a 45° angle extending from said 10% of said thickness to said outer surface of said end structure.

14. The intraluminal expandable medical device according to claim 1, wherein said first plane is defined by a 60° angle extending from said 10% of said thickness to said outer surface of said end structure.

15. The intraluminal expandable medical device according to claim 1, wherein said second plane is defined by a 45° angle extending from said 90% of said thickness to said outer surface of said end structure.

16. The intraluminal expandable medical device according to claim 1, wherein said second plane is defined by a 30° angle extending from said 90% of said thickness to said outer surface of said end structure.

17. The intraluminal expandable medical device according to claim 1, wherein said end structure is a radiopaque marker, said intraluminal expandable medical device is a stent, said stent is self-expanding, and said transitional surface extends at least 60° centered around said end surface around a circumference of said end structure.

18. The intraluminal expandable medical device according to claim 17, wherein said first plane is defined by 30% of said thickness from said outer surface of said end structure, and said second plane is defined by 70% of said thickness from said outer surface of said end structure.

19. The intraluminal expandable medical device according to claim 18, wherein said first plane is defined by a 45° angle extending from said 30% of said thickness to said outer surface of said end structure, and said second plane is defined by a 45° angle extending from said 70% of said thickness to said outer surface of said end structure.

20. The intraluminal expandable medical device according to claim 19, wherein said end structure has a generally elliptical circumference and said transitional surface extends generally elliptically around at least a portion of said circumference of said end structure, and said transitional surface is defined by a generally constant angle as said transitional surface extends around said circumference of said end structure.
